(19) United States
(12) Patent Application Publication
(10) Pub. No.: US 2026/0314983 A1
(43) Pub. Date: Oct. 15, 2026

(54) ATRIAL FLUTTER CLASSIFICATION SYSTEM

(71) Applicants: The Vektor Group, Inc., San Diego, CA (US); The Regents of the University of California, Oakland, CA (US)

(72) Inventors: Christopher J. T. Villongco, San Diego, CA (US); Gordon Ho, San Diego, CA (US); Christian David Marton, San Diego, NJ (US)

(21) Appl. No.: 19/157,347

(22) PCT Filed: Feb. 14, 2024
(86) PCT No.: PCT/US2024/015856

§ 371 Date: Aug. 15, 2025

Related U.S. Application Data
(60) Provisional application No. 63/446,324, filed on Feb. 16, 2023.

(51) Int. Cl. A61B 5/361; A61B 5/332; A61B 5/341; A61B 5/343; G16H 50/20

(52) U.S. Cl. CPC A61B 5/361; A61B 5/332; A61B 5/341; A61B 5/343; G16H 50/20

(57) ABSTRACT

A system for generating a machine learning (ML) model to identify an atrial flutter (AFL) type of a cardiac arrhythmia is provided. For each of a plurality of cardiograms, the system generates training data by identifying one or more portions of that cardiogram that relate to the AFL type to which that cardiogram is mapped. For each of a plurality of the portions, the system generates a feature vector that includes the portion and the additional features and a label that is based on the AFL type. The system trains the ML model using the training data to learn weights for the ML model. The ML model inputs a cardiogram and additional features and outputs an AFL type.

### CROSS-REFERENCE TO RELATED APPLICATION

This application claims the benefit of U.S. Provisional Application No. 63/446,324 entitled “Atrial Flutter Classification System” and filed on Feb. 16, 2023, which is hereby incorporated by reference.

### BACKGROUND

Atrial arrhythmias include coarse atrial fibrillation, macro-reentrant atrial flutters (AFLs), and focal atrial tachycardias. An AFL is a common type of arrhythmia and is characterized by a very rapid heart rate (e.g., cycling) with multiple P waves within an R-R interval. Each P wave represents a contraction (or partial contraction) of an atrium. The atrial heart rate can reach 300 beats per minute. With normal sinus rhythm, a cardiac contraction is initiated in the sinoatrial (SA) node. With AFLs, however, an atrial contraction is initiated by a reentrant circuit within an atrium. An AFL in the right atrium includes a typical cavotricuspid isthmus (CTI) AFL and in the left atrium includes most commonly a mitral annular AFL and a roof AFL.

Although AFLs are generally not life threatening, an AFL may cause chest discomfort, difficulty breathing, and loss of consciousness. AFLs may result in pooling of blood in the atrium due to incomplete contraction/expansion of the atrium which may cause blood clots to form which can lead to a stroke. An AFL may also result in cardiomyopathy.

Treatment of an AFL depends in part on the type of AFL. During an AFL episode, treatments to restore normal sinus rhythm can include administering beta blockers to reduce the heart rate or administering electrical shock to restore normal sinus rhythm. Longer-term treatments include the use of blood thinners. A permanent treatment is an ablation procedure that terminates the function of the tissue of the reentrant circuit resulting in scar tissue.

To treat an AFL with an ablation, it is important to know the type of AFL and its source location. The source location of the source (e.g., reentrant circuit or its exit site) of the AFL needs to be identified so that the ablation can effectively target the treatment of the AFL. An ablation to treat an AFL may employ various technologies, such as radiofrequency energy ablation, pulsed field ablation, other electromagnetic energy ablation, cryoablation, ultrasound ablation, laser ablation, external radiation sources, and so on by targeting a region of the atrium to terminate the AFL. The source location and type of an AFL can be provided to an ablation therapy device (e.g., stereotactic ablative radiotherapy (SABR) device) that controls the performance of the ablation.

### BRIEF DESCRIPTION OF THE DRAWINGS

FIG. 1 is a diagram that illustrates a classification hierarchy of atrial arrhythmias.

FIG. 2 is a block diagram that illustrates an AFL ML architecture in some embodiments.

FIG. 3 is a flow diagram that illustrates a run simulations component of the AFL classification system in some embodiments.

FIG. 4 is a flow diagram that illustrates a generate training data component that generates training data for the AFL ML model in some embodiments.

FIG. 5 is a flow diagram that illustrates a train device AFL ML models component in some embodiments.

FIG. 6 is a flow diagram that illustrates a classify ECG component of the AFL classification system in some embodiments.

FIG. 7 illustrates the initiation of an AFL during a simulation.

### DETAILED DESCRIPTION

An AFL classification system is provided that inputs a cardiogram and identifies the AFL type that the cardiogram represents. In some embodiments, the AFL classification system trains an AFL machine learning (ML) model that inputs a cardiogram and outputs an AFL type. The AFL ML model is trained using training data derived from mappings of a cardiogram (e.g., electrocardiogram (ECG) or vectorcardiogram (VCG)) and optionally additional features to the arrhythmia type (e.g., an AFL type or other arrhythmia type) that the cardiogram represents. For each of the mappings, the AFL classification system identifies cardiogram portions (e.g., an entire cardiogram or an AFL cycle) of that cardiogram that relate to the arrhythmia type to which that cardiogram is mapped. For each of the cardiogram portions, the AFL classification system generates a feature vector that includes a cardiogram portion and optionally additional features and a label that is based on the arrhythmia type. The AFL classification system then trains the AFL ML model using the training data (i.e., the labeled feature vectors) to learn weights for the AFL ML model. After the AFL ML model is trained, the AFL classification system inputs a target cardiogram portion (e.g., collected from a patient) and/or additional features to the AFL ML model which outputs the AFL type that the target cardiogram portion represents. Although the AFL classification system is described primarily as using cardiograms that are ECGs, the AFL system may use other types of cardiograms such as VCGs or body surface potential maps (BSPMs). The AFL ML model may be trained using a voltage-time series and/or an image representation of a cardiogram or a latent vector representation of a cardiogram (e.g., generated by an autoencoder as discussed below). In some embodiments, the AFL ML model may be trained using additional features such as average voltage and principal components that are derived from a cardiogram. Although the AFL ML model could be trained using only these additional derived features, the use of derived features necessarily results in some information loss.

The AFL classification system may employ a variety of ML technologies such as a neural network (NN), a convolutional neural network (CNN), a recurrent neural network (RNN), a transformer, k-means clustering, a support vector machine (SVM), a decision tree, a state-space model, and so on. Some ML technologies that the AFL classification system may employ are described below. The AFL ML model may be a single ML model that inputs an ECG and outputs AFL type. The AFL ML model may also be multiple ML sub-models that are invoked in series to filter out ECGs that do not represent an AFL with a final ML sub-model that inputs an AFL ECG and outputs an AFL type. The ML sub-models may include an arrhythmia ML sub-model that classifies an ECG as an arrhythmia, an atrial ML sub-model that classifies an arrhythmia ECG as atrial, a regular atrial ML sub-model that classifies an atrial ECG as regular atrial, a macro-reentrant AFL ML sub-model that classifies a regular atrial ECG as a macro-reentrant AFL, a left/right atrial ML sub-model that classifies a macro-reentrant ECG as left atrial or right atrial, a right atrial ML sub-model that classifies a right atrial ECG as typical AFL, and a left atrial ML sub-model that classifies a left atrial ECG as mitral annular AFL or roof AFL. The ML sub-models can be trained together using a combined loss function or separately trained using a separate loss function for each ML sub-model.

When additional features are used, various ML architectures may be used for an AFL ML model (or sub-model). For example, an ML architecture may include a CNN that inputs an ECG and a NN (or other classifier such as a support vector machine, decision tree, and so on) that inputs the additional features and inputs the output of the CNN and outputs the AFL type. With such an ML architecture, the CNN and NN may be trained together using a combined loss function or separately using separate loss functions (e.g., train the CNN first and then the NN). If the ECGs are represented as a voltage-time series, an RNN may be used in place of the CNN. As another example, an ML architecture may include an CNN (or RNN) autoencoder to generate a latent vector that represents the input ECG. That latent vector (along with any additional features) may be input to an NN that outputs the AFL type. A feature representing the ECG may alternatively be represented by principal components generated using a Principal Component Analysis (PCA) of the ECG. Another a latent vector representing an ECG may also be generated using a self-supervised ML model based on independent component analysis and contrastive learning. (See, Schneider, S., Lee, J. H. and Mathis, M. W., 2023. Learnable latent embeddings for joint behavioural and neural analysis. Nature, pp. 1-9, which is hereby incorporated by reference.)

In addition, or alternatively, to the use of a raw ECG (e.g., voltage-time series or image) as a feature, the AFL classification system may use additional features such as ECG derived features, cardiac features, and clinical features. The ECG derived features are derived from the ECG and may include, for example, R-R intervals, R-R intervals normalized to a standard length, longest R-R interval, R-R interval length, number of cycles in an R-R interval, average voltage of a cycle, and so on. The cardiac features may include, for example, cardiac images (e.g., CT scans or x-rays), ablation locations, ablation patterns, atrial geometry, presence of atrial fibrosis, and so on. The clinical features may include, for example, age, sex, weight, presence of comorbidities (e.g., coronary artery disease, thyroid disease, and diabetes), and so on.

In some embodiments, a feature may be derived from a VCG. The feature may be the VCG itself, for example, represented as a time series of vectors (magnitude and direction) representing the heart's electrical activity as a dipole. The VCG may be input directly into an ML model (e.g., RNN, CNN, or transformer). A feature derived from a VCG may be, for example, a latent vector generated by an autoencoder similar to that described above for an ECG.

In some embodiments, the training data may be based on clinical data and/or simulated data. The clinical data may be derived from electronic health records (EHRs) and may include, for patients with and without AFL, cardiograms, cardiac characteristics (e.g., prior ablation locations), AFL source location, cardiac images (e.g., CT or x-ray images), and so on.

The simulated data may be generated by simulating electrical activity of simulated hearts with different cardiac characteristics such as electrical characteristics, anatomical characteristics, AFL type, AFL source location, prior ablation locations, scar tissue characteristics, and so on. (See, U.S. Pat. No. 10,319,144 that is entitled “Computational Localization of Fibrillation Sources” and that issued on Jun. 11, 2019, which is hereby incorporated by reference.) A simulated cardiogram is generated for each simulation based on the simulated electrical activity and possibly assuming various thoracic characteristics (see, the '830 patent application described below).

The AFL classification system may also generate features from the simulations. The AFL classification system may generate features using mapping techniques (e.g., machine learning) that are described in the '144 patent and the '754 and '791 patents (see below). The mapping techniques relate to identifying cardiac characteristics given a patient cardiogram. The mapping techniques employ mappings of cardiac characteristics used in a simulation to the simulated cardiogram generated based on the simulated electrical activity. The features may include any of the cardiac characteristics described above such as AFL type and AFL source location. For example, the AFL ML model may be trained with the AFL source location as a feature. To identify the AFL type for a patient, the AFL classification system employs a mapping technique to identify an AFL source location given the patient cardiogram. The AFL classification system then inputs the patient cardiogram and the AFL source location to the AFL ML model to identify an AFL type. When the AFL model includes sub-models, various features may be input to the sub-models. For example, scar tissue characteristics (e.g., location, 3D shape, tissue type such as borderzone) may be input to the macro-reentrant ML sub-model.

The AFL classification system may generate simulated cardiac images based on the three-dimensional (3D) mesh used in the simulation. The simulated cardiac images are generated by projecting simulated radiation through a simulated 3D representation of a heart derived from the 3D mesh onto two-dimensional (2D) slices (e.g., of a simulated CT image). The projecting factors in radiation transmissivity of tissue within the heart (e.g., myocardial tissue and blood tissue. (See, U.S. Pat. No. 10,713,791 that is entitled “Computational Simulations of Anatomical Structures and Body Surface Electrode Positioning and that issued on Jul. 14, 2020, which is hereby incorporated by reference.) The AFL classification system may use features derived from cardiac images in the training and applying of the AFL ML model.

In some embodiments, the AFL classification system may classify ECGs with a subset of the standard 12 leads or with non-standard leads. For example, an ECG may include lead I and leads V1-V6. As another example, an ECG may include a non-standard lead derived from electrodes placed on the upper right and left portions of the chest. When an ECG includes only a subset of the 12 standard leads, the AFL classification system may train an AFL ML model using ECGs with that subset of leads. When an ECG includes non-standard leads, the AFL classification system may generate from the simulations ECGs assuming the placement of electrodes from which those non-standard leads were generated. In such a case, the AFL classification system may generate the non-standard leads from previous simulations of electrical activity without having to run additional simulations. The AFL classification system may employ various techniques to generate a corrected ECG to account for a non-standard placement of electrodes or to convert a non-standard ECG to a standard ECG. Various ECG correction and ECG conversion techniques are described in PCT App. No. PCT/US23/75742 that is entitled “Electrocardiogram Lead Generation” and that was filed on Oct. 2, 2023, which is hereby incorporated by reference.

In some embodiments, the AFL classification system may receive ECGs collected in non-clinical settings and/or with personal ECG collection devices such as smartwatches, Holter monitors, smartphones, and so on. For example, a smartphone may receive, via a Bluetooth or WiFi connection, an ECG collected and transmitted by an ECG patch. The ECGs collected by such collection devices may not represent standard leads or, if they do, may not be an accurate representation of standard leads, for example, because of noise, low resolution, low-quality electrodes, and so on. Moreover, some of the standard leads may actually be derived from leads that are estimated based on collected leads. For example, a non-standard lead may be collected, and a collection device may estimate what a standard lead would be from the non-standard leads (or non-standard electrode placements).

To account for differences in the ECGs received from various collection devices, the AFL classification system may generate a device-specific AFL ML model for different types of collection devices. The AFL classification system may train a device-specific AFL ML model using training data that includes ECGs that are collected from patients using that device and that have been labeled. The ECGs may be labeled manually (e.g., previously by a physician) or automatically. The automatic labeling may involve use of a device lead conversion ML model (described below) to convert to standard leads and the AFL ML model to identify the AFL type that is used as a label.

The training data that is available for a device-specific AFL ML model may not be enough to generate a sufficiently accurate device-specific AFL ML model. To overcome this problem, the AFL classification system may initialize the weights of the device-specific ML model to prior weights of a prior AFL ML model that was not trained using ECGs collected from the device. Such initialization may both speed up the training and improve the accuracy. A prior AFL ML model may be trained using ECGs collected from a different type of device such as a smartphone when the device is a smartwatch. A prior AFL ML model may also be trained using simulated ECGs.

The AFL classification system may employ a device lead conversion ML model that inputs a device lead and outputs a standard lead. To train a device lead conversion ML model, the AFL classification system may access a collection of device leads and corresponding standard leads that are collected from patients. For example, in a clinical setting, a personal collection device may be used to collect device leads while standard leads are simultaneously collected by a clinical collection device. The training data for a device lead conversion ML model may be the device leads labeled with the simultaneously collected standard leads.

Since the training data may be limited for some devices, the AFL classification system may initialize the weights of a device lead conversion ML model to weights of a generic lead conversion ML model that is not device specific or to weights of a device lead conversion ML model for another device. A generic lead conversion ML model may be trained using training data that includes a non-standard lead generated from a simulation labeled with a standard lead generated from the same simulation. The initialization of classifier weights of an ML model is described in U.S. Pat. No. 10,860,754 entitled “Calibration of Simulated Cardiograms” and issued on Dec. 8, 2020, which is hereby incorporated by reference. The AFL classification system effectively “leverages” the pre-trained generic (or other device) lead conversion ML model as described below.

The AFL classification system may also generate additional training data using generative techniques. For example, the AFL classification system may employ a diffusion model or a generative adversarial network (GAN) to generate the additional training data. A diffusion model and a GAN may be trained using simulated and/or clinical data. Once trained the reverse diffusion process of the diffusion model and the generator of the GAN may be used to generate the additional training data.

In some embodiments, the AFL classification system employs simulations of electrical activity of AFLs to generate mappings of cardiograms to AFL source location and/or AFL type. Each simulation is based on a three-dimensional (3D) mesh representing the atria and ventricles or just representing an atrium. The 3D mesh specifies vertices and edges that connect the vertices. Each vertex represents an area within the heart, and each edge indicates that the vertices connected by the edge represent adjacent areas. Each vertex is associated with electrical characteristics and values representing electrical activity within the area that the vertex represents. The values may represent, for example, the action potential over time. To simulate an AFL, an area of slow conduction of electrical activity in an atrium is designated by setting the electrical characteristics of the vertex (or vertices) representing the area to represent conduction that is slower than normal conduction of the surrounding areas in the atrium. Also, a line of block is designated as being adjacent to the area of slow conduction so that electrical activity initiated on the side of the line of block opposite the area of slow conduction (or more generally initiated at a location relative to the line of block and the area of slow conduction) does not propagate through the line of block to the area of slow conduction.

The AFL classification system then runs the simulation using the 3D mesh that includes a representation of the area of slow conduction and the line of block. During simulation intervals (e.g., each representing 1 millisecond), values of vertices of the 3D mesh are updated based on the simulated electrical activity during that interval. During the initial stages of the simulation, an electrical stimulus is applied on the side of the line of block that is opposite the area of slow conduction. Since the line of block prevents the electrical stimulus from passing through the line of block to the area of slow conduction, the electrical activity propagates via normal conduction around the atrium to the other side of the area of slow conduction. The line of block is also removed. The electrical activity propagates slowly through the area of slow conduction and when the electrical activity propagates to where the line of block was, the electrical activity propagates through the area of normal conduction around the atrium back to the area of slow conduction. The electrical activity then propagates through the area of slow conduction, the area of normal conduction, and back to the area of slow conduction represents an atrial cycle. The simulation continues until the AFL has stabilized. A technique for determining the stability of arrhythmia sources is described in Krummen, D. E., Hayase, J., Morris, D. J., Ho, J., Smetak, M. R., Clopton, P., Rappel, W. J. and Narayan, S. M., 2014. Rotor stability separates sustained ventricular fibrillation from self-terminating episodes in humans. Journal of the American College of Cardiology, 63 (24), pp. 2712-2721, which is hereby incorporated by reference. The various types of AFLs can be simulated based on the position of the area of slow conduction within the atrium and the location of the line of block.

In some embodiments, the AFL classification system may employ a bootstrapping technique to reduce the time needed to run the simulations. The AFL classification system may identify groups of simulated hearts with cardiac characteristics that are similar, for example, based on electrical characteristics and/or anatomical characteristics and AFL type and AFL source location. For each group, the AFL classification system selects a simulated heart and runs a simulation based on the cardiac characteristics of that simulated heart. The AFL classification system runs an initial simulation that includes adding a line of block, applying an electrical stimulus, removing the line of block, and continuing the simulation at least until the electrical activity of the AFL stabilizes. To run a simulation for each of the other simulated hearts, the AFL classification system generates a 3D mesh based on the electrical characteristics and anatomical characteristics of that simulated heart. The AFL classification system also initializes the values of the 3D mesh representing the electrical activity (e.g., action potential) to the values of the initial simulation after the AFL had stabilized. The AFL classification system then runs the simulation for that simulated heart. Because the initialization is based on the initial simulation, the AFL of that simulation stabilizes faster than if the values were initialized to random or default values thus speeding up the simulations.

The AFL classification system may also employ a bootstrapping technique using patient-specific cardiac characteristics. For example, in preparation for an ablation procedure, a medical provider may want to analyze the patient's arrhythmia. In such a case, a patient's cardiac characteristics may be collected. The medical provider may employ the AFL classification system to run patient-specific simulations assuming various AFL types and source locations. Before running a patient-specific simulation assuming an AFL type and an AFL source location, the AFL classification system identifies a simulation based on simulated cardiac characteristics that are similar to those of the patient and with an AFL type and AFL source location of the patient-specific simulation. To run the patient-specific simulation, the AFL classification system initializes the values of a patient-specific 3D mesh representing the electrical activity to that of the identified simulation after the AFL had stabilized. The AFL classification system can run patient-specific simulations for various AFL types and AFL source locations. A simulated cardiogram can be generated for each patient-specific simulation and compared to the patient's cardiogram. The AFL type and AFL source location of the simulation with the best matching simulated cardiogram may be informative of the patient's AFL type and AFL source location.

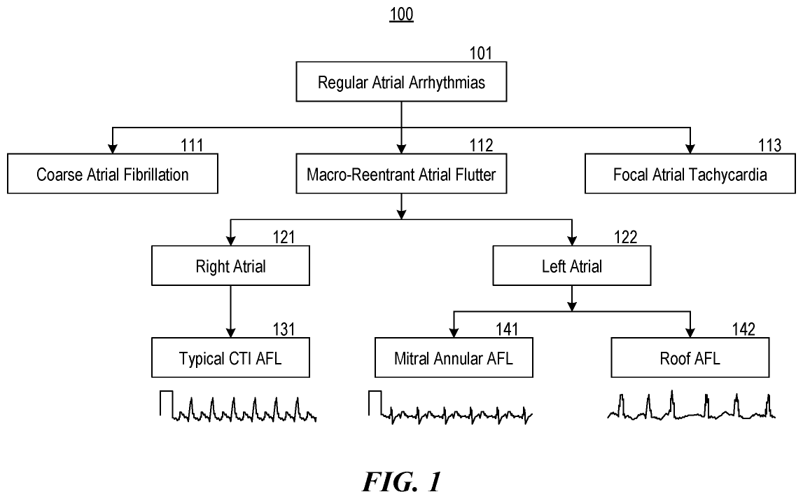

The AFL classification system runs simulations for the different types of AFLs. FIG. 1 is a diagram that illustrates a classification hierarchy of AFLs. Block 101 of the classification hierarchy 100 represents regular arrhythmias. A regular atrial arrhythmia is an abnormal heart rhythm that originates from an atrium and maintains a consistent regular rhythm. Blocks 111, 112, and 113 represent coarse atrial fibrillations, macro-reentrant AFLs, and focal atrial tachycardias, respectively. A coarse atrial fibrillation is caused by a rotor which is a rapidly spinning circle of electrical activity around scar tissue. A macro-reentrant AFL is caused by a reentrant circuit in which electrical activity continuously travels in loop. A reentrant circuit may include an isthmus (an area of slow conduction) between scar tissue with an entrance site and an exit site. The electrical activity enters the isthmus at the entrance site, travels from the entrance site through the isthmus to the exit site, and travels from the exit site around the scar tissue to the entrance site forming the loop. A micro-reentrant AFL, in contrast to a macro-reentrant AFL, has attributes that are similar to a focal source. A focal atrial tachycardia is a type of supraventricular tachycardia in which rapid and regular heart beats originate from a single site (referred to as a focus). Blocks 121 and 122 represent macro-reentrant AFLs of the right atrium and left atrium, respectively. Block 131 represents a typical CTI AFL (also known as a typical AFL). A typical CTI AFL is a reentrant circuit with a path that includes the cavotricuspid isthmus. The cavotricuspid isthmus is a narrow band of tissue between the inferior vena cava and the tricuspid valve. An atypical AFL, in contrast, is located outside of and does not involve the cavotricuspid isthmus. Blocks 141 and 142 represent mitral annular AFLs and roof AFLs of the left atrium, respectively. A mitral annular AFL (which may be considered an atypical flutter) originates from a reentrant circuit in which electrical activity travels around the mitral valve annulus. A roof AFL (which may be considered an atypical flutter) originates from a reentrant circuit in which electrical activity circulates in a pathway that connects the left and right pulmonary veins along the roof of the left atrium. In some embodiments, the AFL classification system inputs an ECG and outputs the classification of the ECG as a typical CTI AFL, a mitral annular AFL, and a roof AFL.

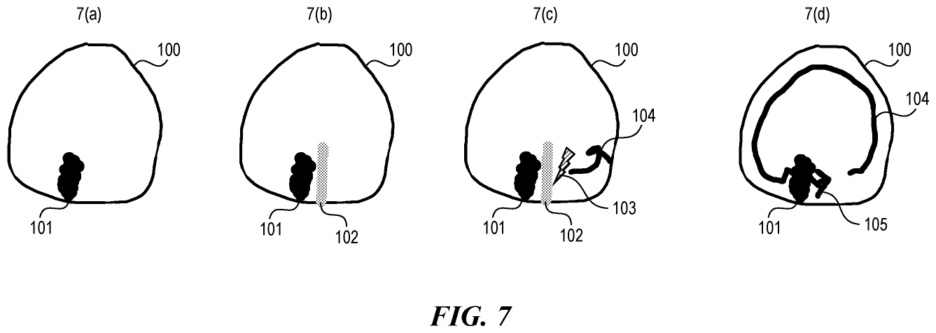

FIG. 7 illustrates the initiation of an AFL during a simulation. Referring to FIG. 7(a), an area of slow conduction 101 is added to a 3D mesh 100 representing an atrium. The rest of the atrium represents normal conduction. Referring to FIG. 7(b), a line of block 102 that is adjacent to the area of slow conduction 101 is then added to the 3D mesh 100. Referring to FIG. 7(c), an electric stimulus is then applied to an electrical stimulus area 103 of the 3D mesh 100 on the side of the line of block 102 that is opposite of the area of slow conduction 101. The electrical stimulus initiates propagation of electrical activity 104 around the atrium to the area of slow conduction 101. Referring to FIG. 7(d), the line of block 102 is removed so that the propagation of electrical activity 104 can continue through the area of slow conduction 101 and exit 105 to initiate an AFL cycle. Since the area of slow conduction covers the CTI and the line of block is to the right of the area of slow conduction 101, the resulting AFL is a typical CTI AFL with a counterclockwise propagation direction. If the line of block were placed on the opposite of the area of slow conduction 101, the resulting AFL would be a reverse typical CTI AFL with a clockwise propagation direction. Mechanisms for various types of AFLs are described in Cosio, F. G., 2017. Atrial flutter, typical and atypical: a review. Arrhythmia & electrophysiology review, 6 (2), p. 55, which is hereby incorporated by reference. Atypical AFL may be simulated by placing the area of slow conduction and the line of block at various locations. For example, a roof AFL may be simulated by placing the area of slow conduction and the line of block at the pathway between the left and right pulmonary veins.

The simulation results in a value for each vertex at each simulation interval. From those values, the AFL classification system generates a simulated cardiogram that represents an AFL. The AFL classification system runs simulations assuming different simulation characteristics such as cardiac geometries, atrial electrical characteristics (e.g., action potential), areas of slow conduction, lines of block, AFL type, and so on. Mappings of the simulated cardiograms to their corresponding simulation characteristic may be used to identify the source location of an AFL and an AFL type based on similarity between a simulated cardiogram and a patient cardiogram. ML models may be trained using the mappings to identify a source location and/or AFL type.

The AFL classification system may provide an application programming interface (API) through which a collection device may provide an ECG and an identifier of the person from which the ECG was collected. The AFL classification system may input the ECG into an AFL ML model to identify an AFL type. The AFL classification system may submit the ECG and AFL type to an electronic health record (EHR) system to add to the person's EHR. The AFL classification system may also notify the collection device of the AFL type for presentation to the person. The AFL classification system may be used in real time to classify ECGs as they are collected (e.g., in a clinical setting or during an electrophysiology procedure) or in batch mode to classify ECGs that were previously stored in an EHR system.

A machine learning (ML) model employed by the AFL classification system may be any of a variety or combination of supervised, semi-supervised, self-supervised, or unsupervised ML models including a neural network such as fully connected, convolutional, recurrent, or autoencoder neural network, or restricted Boltzmann machine, a support vector machine, a Bayesian classifier, k-means clustering, and so on. When the ML model is a deep neural network, the model is trained using training data that includes features derived from data and labels corresponding to the data (e.g., ECGs labeled with source location and/or AFL type. The training results in a set of weights for the activation functions of the layers of the deep neural network. The trained deep neural network can then be applied to new data (e.g., a patient ECG) to generate a label for that new data (e.g., source location). When the ML model is a support vector machine, a hyper-surface is found to divide the space of possible inputs. For example, the hyper-surface attempts to split the positive examples (e.g., typical CTI AFLs) from the negative examples (e.g., roof AFL) by maximizing the distance between the nearest of the positive and negative examples to the hyper-surface. The trained support vector machine can then be applied to new data to generate a classification (e.g., typical CTI AFL or not) for the new data. An ML model may generate values of discrete domain (e.g., classification), probabilities, and/or values of a continuous domain (e.g., regression value, classification probability).

Various techniques can be used to train a support vector machine such as adaptive boosting, which is an iterative process that runs multiple tests on a collection of training data. Adaptive boosting transforms a weak learning algorithm (an algorithm that performs at a level only slightly better than chance) into a strong learning algorithm (an algorithm that displays a low error rate). The weak learning algorithm is run on different subsets of the training data. The algorithm concentrates increasingly on those examples in which its predecessors tended to show mistakes. The algorithm corrects the errors made by earlier weak learners. The algorithm is adaptive because it adjusts to the error rates of its predecessors. Adaptive boosting combines rough and moderately inaccurate rules of thumb to create a high-performance algorithm. Adaptive boosting combines the results of each separately run test into a single, very accurate classifier. Adaptive boosting may use weak classifiers that are single-split trees with only two leaf nodes.

A neural network model has three major components: architecture, loss function, and search algorithm. The architecture defines the functional form relating the inputs to the outputs (in terms of network topology, unit connectivity, and activation functions). The search in weight space for a set of weights that minimizes the loss function is the training process. A neural network model may use a radial basis function (RBF) network and a standard or stochastic gradient descent as the search technique with backpropagation.

A convolutional neural network (CNN) has multiple layers such as a convolutional layer, a rectified linear unit (ReLU) layer, a pooling layer, a fully connected (FC) layer, and so on. Some more complex CNNs may have multiple convolutional layers, pooling layers, and FC layers. Each layer includes a neuron for each output of the layer. A neuron inputs outputs of prior layers (or original input) and applies an activation function to the inputs to generate an output.

A convolutional layer may include multiple filters (also referred to as kernels or activation functions). A filter inputs a convolutional window, for example, of an image, applies weights to each pixel of the convolutional window, and outputs value for that convolutional window. For example, if the static image is 256 by 256 pixels, the convolutional window may be 8 by 8 pixels. The filter may apply a different weight to each of the 64 pixels in a convolutional window to generate the value.

An activation function has a weight for each input and generates an output by combining the inputs based on the weights. The activation function may be a rectified linear unit (ReLU) that sums the values of each input times its weight to generate a weighted value and outputs max (0,weighted value) to ensure that the output is not negative. The weights of the activation functions are learned when training an ML model. The ReLU function of max (0,weighted value) may be represented as a separate ReLU layer with a neuron for each output of the prior layer that inputs that output and applies the ReLU function to generate a corresponding “rectified output.”

A pooling layer may be used to reduce the size of the outputs of the prior layer by downsampling the outputs. For example, each neuron of a pooling layer may input 16 outputs of the prior layer and generate one output resulting in a 16-to-1 reduction in outputs.

An FC layer includes neurons that each input all the outputs of the prior layer and generate a weighted combination of those inputs. For example, if the penultimate layer generates 256 outputs and the FC layer inputs a neuron for each of three classifications (e.g., course AF, macro-reentrant AFL, and focal atrial tachycardia), each neuron inputs the 256 outputs and applies weights to generate value for its classification.

Multimodal machine learning combines different modalities of input data to make a prediction. The modalities may be, for example, an ECG image, cardiac geometric characteristics (e.g., atrial dimensions), scar tissue locations, and so on.

In one multimodal ML approach, referred to as “early fusion,” data of the different modalities is combined at the input stage and is then trained on the multimodal data. The training data for these modalities includes a collection of sets of containing features derived from the different modalities and labels. A modality may be used in its original form or preprocessed, for example, to reduce its dimensionality by compressing the data into byte arrays or applying a principal component analysis. The concatenated bytes may be then processed by a cross-attention mechanism to condense the concatenated bytes into a vector of a fixed size. The vectors are then used to train an ML model primarily using supervised approaches although self-supervised or unsupervised approaches may also be used.

In a second multimodal ML approach, data from different modalities may be kept separate at the input stage and used as inputs to different, modality-specific ML models (e.g., a CNN for an ECG image and a neural network for scar tissue locations). The modality-specific ML models may be trained jointly such that information from across different modalities is combined to make predictions, and the combined (cross-modality) loss is used to adjust model weights. Alternatively, the modality-specific ML models may also be trained separately using a separate loss function for each modality. A combined ML model is then trained based on the outputs of the modality specific models. Continuing with the example, the training data for each modality-specific ML model may be based on its data along with a label. The combined ML model is then trained with the outputs of the modality-specific ML models with a final label.

Transformer machine learning was introduced as an alternative to a recurrent neural network that is both more effective and more parallelizable. (See, Vaswani, A., Shazeer, N., Parmar, N., Uszkoreit, J., Jones, L., Gomez, A. N., Kaiser, Ł. and Polosukhin, I., 2017. Attention is all you need. Advances in neural information processing systems, 30, which is hereby incorporated by reference.) Transformer machine learning was originally described in the context of natural language processing (NLP) but has been adapted to other applications such as image processing to augment or replace a CNN. In the following, transformer machine learning is described in the context of NLP as introduced by Vaswani.

A transformer includes an encoder whose output is input to a decoder. The encoder includes an input embedding layer followed by one or more encoder attention layers. The input embedding layer generates an embedding of the inputs. For example, if a transformer ML model is used to process a sentence as described by Vaswani, each word may be represented as a token that includes an embedding of a word and its positional information. Such an embedding is a vector representation of a word such that words with similar meanings are closer in the vector space. The positional information is based on the position of the word in the sentence.

The first encoder attention layer inputs the embeddings and the other encoder attention layers input the output from the prior encoder attention layer. An encoder attention layer includes a multi-head attention mechanism followed by a normalization sublayer whose output is input to a feedforward neural network followed by a normalization sublayer. A multi-head attention mechanism includes multiple self-attention mechanisms that each inputs the encodings of the previous layer and weighs the relevance encodings to other encodings. For example, the relevance may be determined by the following attention function:

$$Attention ( Q , K , V ) = softmax ( \frac{{QK}^{T}}{\sqrt{{d}_{k}}} ) V$$

where Q represents a query, K represents a key, V represents a value, and dk represents the dimensionality of K. This attention function is referred to as scaled dot-product attention. In Vaswani, the query, key, and value of an encoder multi-head attention mechanism is set to the input of the encoder attention layer. The multi-head attention mechanism determines the multi-head attention as represented by the following:

$$MultiHead ( Q , K , V ) = concat ( {head}_{1} , … , {head}_{8} ) {W}^{o}$$

$${head}_{i} = Attention ( {QW}_{i}^{Q} , {KW}_{i}^{K} , {VW}_{i}^{V} )$$

where W represents weights that are learned during training. The weights for the feedforward networks are also learned during training. The weights may be initialized to random values. A normalization layer normalizes its input to a vector having a dimension as expected by the next layer or sub-layer.

The decoder includes an output embedding layer, decoder attention layers, a linear layer, and a softmax layer. The output embedding layer inputs the output of the decoder shifted right. Each decoder attention layer inputs the output of the prior decoder attention layer (or the output embedding layer) and the output of the encoder. The embedding layer is input to the decoder attention layer, the output of the decoder attention layer is input the linear layer, and the output of the linear layer is input to the softmax layer which outputs probabilities. A decoder attention layer includes a decoder masked multi-head attention mechanism followed by a normalization sublayer, a decoder multi-head attention mechanism followed by a normalization sublayer, and a feedforward neural network followed by a normalization sublayer. The decoder masked multi-head attention mechanism masks the input so that predictions for a position are only based on outputs for prior positions. A decoder multi-head attention mechanism inputs the normalized output of the decoder masked multi-head attention mechanism as a query and the output of the encoder as a key and a value. The feedforward neural network inputs the normalized output of the decoder multi-head attention mechanism. The normalized output of the feedforward neural network is the output of that multi-head attention layer. The weights of the linear layer are also learned during training.

After being trained, a sentence may be input to encoder to generate an encoding of the sentence that is input to the decoder. Initially, the output of the decoder that is input to the decoder is set to null. The decoder then generates an output based on the encoding and the null input. The output of the decoder is appended to the decoder's current input, and the decoder generates a new output. This decoding process is repeated until the encoder generates a termination symbol. If the transformer is trained with English sentences labeled with French sentences, then a termination symbol is added to the end of the French sentences. When translating a sentence, the transformer terminates its translation when the termination symbol is generated indicating the end of the French sentence that is completion of the translation.

Although initially developed to process sentences, transformers have been adapted for image recognition. The input a decoder of a transformer may be a representation of fixed-size patches of the image. (See, Dosovitskiy, A., Beyer, L., Kolesnikov, A., Weissenborn, D., Zhai, X., Unterthiner, T., Dehghani, M., Minderer, M., Heigold, G., Gelly, S. and Uszkoreit, J., 2020. An image is worth 16×16 words: Transformers for image recognition at scale. arXiv preprint arXiv:2010.11929, which is hereby incorporated by reference.) The representation of a patch may be, for each pixel of the patch, an encoding of its row, column, and color. The output of the encoder is fed into neural network to generate a classification of the image.

In some embodiments, the AFL classification system may also employ a state space model (SSM) to generate a latent representation of a cardiogram. An example of an SSM is S4 as described in Gu, A. and Dao, T., 2023. Mamba: Linear-time sequence modeling with selective state spaces. arXiv preprint arXiv:2312.00752 (Mamba), which is hereby incorporated by reference. Mamba provides a unique selection mechanism that adapts structured SSM parameters based on the input to selectively focus on relevant information within sequences, effectively filtering out less pertinent information. Mamba integrates SSM with multi-layer perceptron (MLP) blocks to support sequence modeling for sequential data such as cardiograms.

In some embodiments, the AFL classification system may employ a kNN model that provides information relating to an entity. The training data for a kNN model may be training feature vectors (e.g., ECG images) and a label for each feature vector indicating information relating to an entity (e.g., typical CTI AFL) having the values of the features of that feature vector. A kNN model may be used without a training phase that is without learning weights or other parameters to represent the training data. In such a case, the patient feature vector is compared to the training feature vectors to identify a number (e.g., represented by the “k” in kNN) of similar training feature vectors. Once the number of similar training feature vectors are identified, the labels associated with the similar training feature vectors are analyzed to provide information for the entity. The labels of the training feature vectors that are more similar to an entity feature vector may be given a higher weight than those that are less similar. For example, if k is 10 and four training feature vectors are very similar and six are less similar, similarity weights of 0.9 may be assigned to the very similar training feature vectors and 0.2 to the less similar. If three of the four and one of the six have the same information, then the information for the entity is primarily based on that information even though most of the 10 have different information. Conceptually, training feature vectors that are very similar are closer to the entity feature vector in a multi-dimensional space of features and a similarity weight is based on distance between the feature vectors. Various techniques may be employed to calculate a similarity metric indicating similarity between a candidate feature vector and a training feature vector such as a dot product, cosine similarity, a Pearson's correlation, and so on.

In some embodiments, the AFL classification system may employ an ML decision tree. The ML decision tree defines arrhythmia sub-classifications that are associated with cardiograms have certain characteristics. An ML decision may have the same form as a manually generated decision tree. However, the feature associated with each decision node of an ML decision tree may be selected automatically based on analysis of the training data. Each decision node (i.e., non-leaf node) of a decision tree corresponds to a feature and each branch from a node may correspond to a value or range of values for the feature of that decision node. For example, a decision node corresponding to cycle length may have branches for very short, short, medium, and long, and a decision node corresponding to R-R interval may have branches for ranges of average voltage. The leaf nodes of the decision tree indicate each indicate a sub-classification.

For an ML decision tree, an entropy score may be used by an ML decision tree generator to select the feature to be associated with each node. The entropy score for a possible feature for a node is based on the distribution of its values in node feature vectors for that node. A node feature vector for a node has the values of the branches along the path from the root node to that node. If a first possible feature for a node has an equal number of node feature vectors for each value, the entropy for the first possible feature is considered to be high. In contrast, if a second possible feature has node feature vectors for which 75% have the same value, then the entropy for the second possible feature is considered to be low. In such a case, the ML decision tree generator would select the second possible feature for that node. The ML decision tree generator may also analyze features with continuous values, such as cycle length, to identify Fayyad, U. M. and Irani, K. B., 1992. On the handling in decision tree of continuous-valued attributes generation. Machine Learning, 8, pp. 87-102, which is hereby incorporated by reference.

An ML decision tree generator may employ a depth-first, recursive algorithm to build the ML decision tree. The ML decision tree generator may employ a path termination criterion to determine when to terminate a path. The path termination criterion may be, for example, when the percentage of node feature vectors that are associated with the same sub-classification is above a threshold percentage. For example, if 99% of the node feature vectors have sub-classification of macro-reentrant AFL, the ML decision tree generator may add a leaf node indicating macro-reentrant AFL. Other termination criteria may be that the number of node feature vectors is below a threshold number or that the path has reached a maximum depth. In such cases, the ML decision tree generator may add a leaf node that indicates that a sub-classification cannot be provided or that the sub-classification has a low confidence.

If the number of training feature vectors is large, various techniques may be employed to effectively “compress” the training data during a training phase. For example, a clustering technique may be employed to identify clusters of training feature vectors that are similar and have the same label. A training feature vector may be generated for each cluster (e.g., one from the cluster or one based on mean values for the features) as a cluster feature vector and assign a cluster weight to it based on number of training feature vectors in the cluster.

In some embodiments, the AFL classification system may employ a generative adversarial network (GAN) or an attribute (attGAN) to generating training data. (See, He, Z., Zuo, W., Kan, M., Shan, S. and Chen, X., 2019. Attgan: Facial attribute editing by only changing what you want. IEEE transactions on image processing, 28 (11), pp. 5464-5478, and Goodfellow, I., Pouget-Abadie, J., Mirza, M., Xu, B., Warde-Farley, D., Ozair, S., Courville, A. and Bengio, Y., 2014. Generative adversarial nets. Advances in neural information processing systems, 27, which are hereby incorporated by reference.) A GAN employs a generator and discriminator and is trained using real data (e.g., clinical or simulated ECG images or VCGs) as training data. The generator generates generated data based on random input. The generator is trained to generate generated data that cannot be distinguished from the training data. The discriminator indicates whether an input is real or generated. The generator and discriminator are trained in parallel to learn weights. The generator is trained to generate increasingly more realistic generated data, and the discriminator is trained to discriminate between real data and generated data more effectively. After being trained, the generator can be used to generate generated ECGs or VCGs that are realistic. Thus, once trained additional training data for the ML models of the AFL classification system can be generated.

In some embodiments, the AFL classification system may employ a diffusion ML model to generate additional training data using a generative process. (See, Rombach, R., Blattmann, A., Lorenz, D., Esser, P. and Ommer, B., 2022. High-resolution image synthesis with latent diffusion models. In Proceedings of the IEEE/CVF conference on computer vision and pattern recognition (pp. 10684-10695), which is hereby incorporated by reference.) A diffusion ML model is a generative ML model that inputs noisy data and progressively denoises the data until the denoised data appears to be indistinguishable from real data such as an image of an ECG. A diffusion ML model is trained using a forward diffusion process that successively adds noise to input training data such as ECG images to generate noisy data and a reverse diffusion process that successively denoises the noisy data to generate denoised data that approximates the input training data. The training learns weights for the reverse diffusion process that tend to minimize the difference between the input training data and the denoised data. After a diffusion model is trained, the reverse diffusion process is employed to generate data that can be used to train the AFL ML model. To do so, randomly generated noisy data is input into the reverse diffusion process which denoises the noisy data to generate the noised data that appears to real data.

The forward diffusion process employs a Markov chain that incrementally adds Gaussian noise to the training data over a series of steps. This process transforms the training data from its initial distribution to a Gaussian distribution. The reverse diffusion process employs a neural network to incrementally approximate and remove the noise that was added at each step of the forward diffusion process. When generating data, randomly generated noisy data is input into the reverse diffusion process which incrementally removes the noise that was learned during training.

The forward diffusion process systematically adds Gaussian noise to the original data x₀ Gaussian noise over T timesteps, resulting in a sequence of increasingly noisy data x₁, x₂, . . . , x_{T}. The process at each time step t may be represented by the equation:

$${x}_{t} = \sqrt{{α}_{t}} {x}_{t - 1} + \sqrt{1 - {α}_{t}} {ϵ}_{t} , {ϵ}_{t} \sim 𝒩 ( 0 , I )$$

where xₜ is data at timestep t, εₜ is Gaussian noise, αₜ is the amount of noise added, and I is the identity matrix.

The reverse diffusion process learns the distribution of the training data by starting from noise and progressively denoising it over the timesteps. The training estimates the reverse of the forward diffusion process using a neural network that may be represented by the equation:

$${x}_{t - 1} = \frac{1}{\sqrt{{α}_{t}}} ( {x}_{t} - \frac{1 - {α}_{t}}{\sqrt{1 - {α}_{t}}} {f}_{θ} ( {x}_{t} , t ) ) where {\tilde{α}}_{t} = {\prod }_{i = 1}^{t} {α}_{t}$$

represent the cumulative noise and f₀(xₜ, t) represents the neural network.

The goal of training a diffusion model is to minimize the difference between the original data and the data reconstructed by the reverse diffusion process using a loss function that may be represented by the equation.

$$L ( θ ) = {𝔼}_{{x}_{θ} , {ϵ}_{t} , t} [ {‖ {ϵ}_{t} - {f}_{θ} ( {x}_{t} , t ) ‖}^{2} ] .$$

A diffusion model may also include a conditioning mechanism that allows for factoring in a domain-specific information into the reverse diffusion process. The domain-specific information may be employed by a cross-attention mechanism of the neural network (e.g., U-Net architecture) of the reverse diffusion process. The AFL classification system may train the reverse diffusion process with domain-specific information that includes the AFL type and/or the AFL source location. To generate ECG images, the AFL classification system inputs a noisy image and an AFL type into the reverse diffusion process which generates an ECG image corresponding to that AFL type.

To generate images such as images of ECGs, a diffusion ML model may operate on latent vectors representing the images. During training, an autoencoder may be employed to convert the training images, for example, using an encoder of CNN autoencoder (e.g., U-net architecture) to generate latent vectors which are input to the forward diffusion process. The reverse diffusion process generates latent vectors that are input to the decoder of the autoencoder to generate images. Once trained, the latent vector generated by the reverse diffusion process is input to the decoder to generate an image. The autoencoder may be trained separately from the diffusion ML model or trained with the diffusion ML model using a combined loss function.

The computing systems (e.g., network nodes or collections of network nodes) on which the AFL classification system and the other described systems may be implemented may include a central processing unit, input devices, output devices (e.g., display devices and speakers), storage devices (e.g., memory and disk drives), network interfaces, graphics processing units, communications links (e.g., Ethernet, Wi-Fi, cellular, and Bluetooth), global positioning system devices, and so on. The input devices may include keyboards, pointing devices, touch screens, gesture recognition devices (e.g., for air gestures), head and eye tracking devices, microphones for voice recognition, and so on. The computing systems may include high-performance computing systems, distributed systems, cloud-based computing systems, client computing systems that interact with cloud-based computing system, desktop computers, laptops, tablets, e-readers, personal digital assistants, smartphones, gaming devices, servers, and so on. The computing systems may access computer-readable media that include computer-readable storage mediums and data transmission mediums. The computer-readable storage mediums are tangible storage means that do not include a transitory, propagating signal. Examples of computer-readable storage mediums include memory such as primary memory, cache memory, and secondary memory (e.g., DVD), and other storage. The computer-readable storage media may have recorded on them or may be encoded with computer-executable instructions or logic that implements the AFL classification system and the other described systems. The data transmission media are used for transmitting data via transitory, propagating signals or carrier waves (e.g., electromagnetism) via a wired or wireless connection. The computing systems may include a secure crypto processor as part of a central processing unit (e.g., Intel Secure Guard Extension (SGX)) for generating and securely storing keys and for encrypting and decrypting data using the keys and for securely executing all or some of the computer-executable instructions of the AFL classification system. Some of the data sent by and received by the AFL classification system may be encrypted, for example, to preserve patient privacy (e.g., to comply with government regulations such the European General Data Protection Regulation (GDPR) or the Health Insurance Portability and Accountability Act (HIPAA) of the United States). The AFL classification system may employ asymmetric encryption (e.g., using private and public keys of the Rivest-Shamir-Adleman (RSA) standard) or symmetric encryption (e.g., using a symmetric key of the Advanced Encryption Standard (AES)).

The one or more computing systems may include client-side computing systems and cloud-based computing systems (e.g., public or private) that each execute computer-executable instructions of the AFL classification system. A client-side computing system may send data to and receive data from one or more servers of the cloud-based computing systems of one or more cloud data centers. For example, a client-side computing system may send a request to a cloud-based computing system to perform tasks such as running a patient-specific simulation of electrical activity of a heart or training a patient-specific ML model. A cloud-based computing system may respond to the request by sending to the client-side computing system data derived from performing the task such as a source location of an arrhythmia. The servers may perform computationally expensive tasks in advance of processing by a client-side computing system such as training a machine learning model or in response to data received from a client-side computing system. A client-side computing system may provide a user experience (e.g., user interface) to a user of the AFL classification system. The user experience may originate from a client computing device or a server computing device. For example, a client computing device may generate a patient-specific graphic of a heart and display the graphic. Alternatively, a cloud-based computing system may generate the graphic (e.g., in a Hyper-Text Markup Language (HTML) format or an extensible Markup Language (XML) format) and provide it to the client-side computing system for display. A client-side computing system may also send data to and receive data from various medical devices such as an ECG monitor, an ablation therapy device, an ablation planning device, and so on. The data received from the medical devices may include an ECG, actual ablation characteristics (e.g., ablation location and ablation pattern), and so on. The data sent to a medical device may include data, for example, data in a Digital Imaging and Communications in Medicine (DICOM) format. A client-side computing device may also send data to and receive data from medical computing systems that store patient medical history data, descriptions of medical devices (e.g., type, manufacturer, and model number) of a medical facility, that store, medical facility device descriptions, that store results of procedures, and so on. The term cloud-based computing system may encompass computing systems of a public cloud data center provided by a cloud provider (e.g., Azure provided by Microsoft Corporation) or computing systems of a private server farm (e.g., operated by the provider of the AFL classification system.

The AFL classification system and the other described systems may be described in the general context of computer-executable instructions, such as program modules and components, executed by one or more computers, processors, or other devices. Generally, program modules or components include routines, programs, objects, data structures, and so on that perform tasks or implement data types of the AFL classification system and the other described systems. Typically, the functionality of the program modules may be combined or distributed as desired in various examples. Aspects of the AFL classification system and the other described systems may be implemented in hardware using, for example, an application-specific integrated circuit (ASIC) or a field programmable gate array (FPGA).

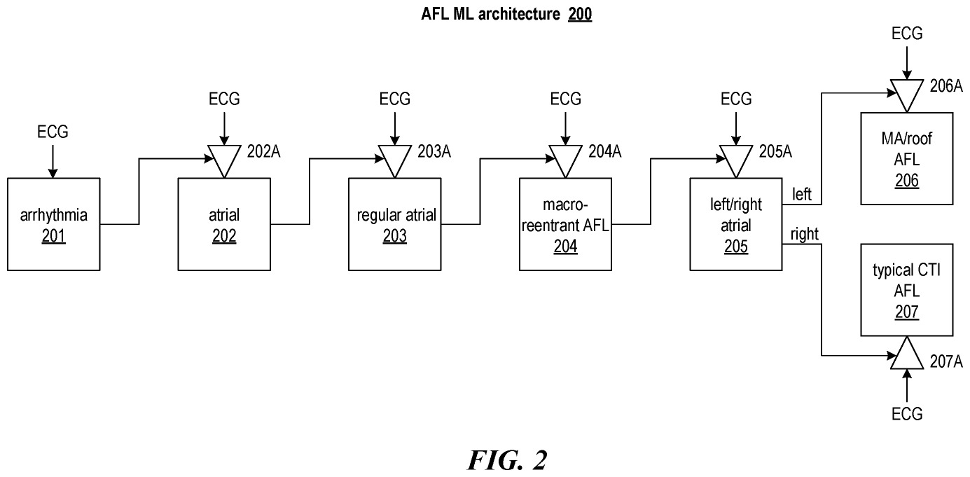

FIG. 2 is a block diagram that illustrates an AFL ML architecture in some embodiments. The AFL ML architecture 200 represents an overall AFL ML model that includes ML sub-models for different classification types. The overall AFL ML architecture inputs a cardiogram (e.g., ECG) and optionally additional features (not shown) and outputs an AFL type. The ML sub-models include an arrhythmia ML sub-model 201, an atrial sub-model 202, a regular atrial sub-model 203, a macro-reentrant AFL sub-model 204, a left/right atrial sub-model 205, an MA/roof sub-model 206, and a typical CTI AFL sub-model 207. Each of the ML sub-models inputs a cardiogram (or portion of a cardiogram selected based on the type of classification of the ML sub-model) and may also input various other features (e.g., cardiac characteristics) and outputs an indicator whether the ECG has the classification of the ML sub-model (e.g., represented by a 0 or 1). The activation controllers 202A, 203A, 204A, 205A, 206A, and 207A input a controller line and an ECG and provide the ECG to the ML sub-model when the controller line is activated. A controller line is activated when the ML sub-model indicates that the ECG is of the ML sub-model type. For example, when the ECG is regular atrial as indicated by regular atrial ML sub-model 203, the controller line that is output is input to activation controller 204A. The activation controller 204 is activated and the macro-entrant AFL ML sub-model 204 classifies the ECG. The ML sub-models of the AFL model represent a hierarchy of levels of ML sub-models of sub-classifications of a cardiogram. In FIG. 2, the arrhythmia ML sub-model is at the top level of the hierarchy, and the MA/roof AFL ML sub-model and the typical CTI AFL ML sub-model are at the bottom level of the hierarchy. Each ML sub-model inputs a cardiogram with the sub-classification of the next higher level. For example, the macro-reentrant ML sub-model classifies a cardiogram that is classified as regular atrial arrhythmia by the regular atrial ML sub-model.

The AFL ML architecture may alternatively combine two or more ML sub-models of FIG. 2 as a combined ML sub-model. For example, ML sub-models 201-203 may be combined into a combined ML sub-model that inputs any type of ECG and outputs an indication of whether the ECG represents a regular atrial arrhythmia. As another example, the ML sub-models 204, 205, and 206 may be combined into a combined ML sub-model that inputs an ECG that represents a regular arrhythmia and outputs an AFL type. The AFL ML model and the ML sub-models may output a classification or output a probability for a classification (e.g., probabilistic classification).

In some embodiments, the AFL classification system identifies AFL cycles of a cardiogram and inputs each cycle to the AFL ML model to generate a classification for that cycle. The AFL classification system may then generate an overall classification based on the classifications of the cycles. For example, if 90% of the cycles are classified as roof AFL, then the overall classification may be roof AFL. Alternatively, the AFL classification system may identify a portion of an R-R interval that represents a sequence of cycles and input that sequence into an ML sub-model. Different ML sub-models may input an entire ECG or different portions of an ECG such as the arrhythmia ML sub-model inputting an R-R interval and the typical CTI AFL sub-model inputting an AFL cycle.

In some embodiments, the AFL classification system accesses a mapping system to map a cycle to a source location or a sequence of cycles to a source location. A mapping system is described in U.S. Pat. No. 10,860,754 that is entitled “Calibration of Simulated Cardiograms” and that issued on Dec. 8, 2020, which is hereby incorporated by reference. The source location may be displayed as a target for an ablation procedure or output to an ablation device (e.g., a stereotactic ablation radiotherapy device).

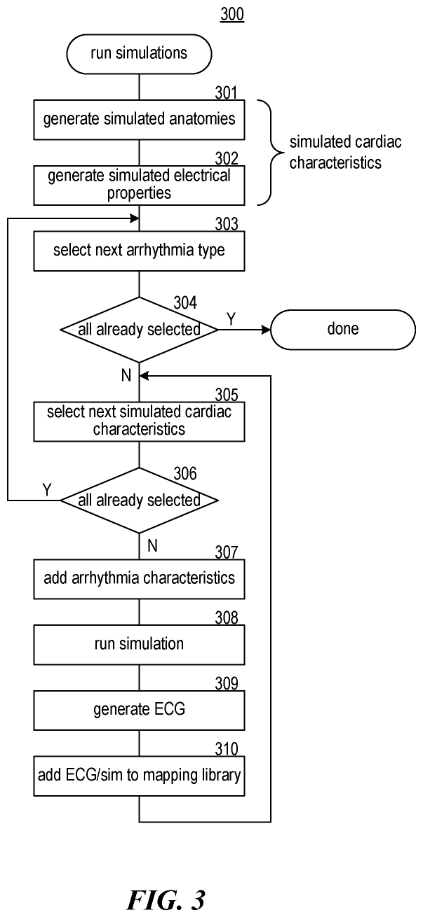

FIG. 3 is a flow diagram that illustrates a run simulations component of the AFL classification system in some embodiments. The run simulations component 300 runs simulations of electrical activity of a heart assuming sets of cardiac characteristics. The cardiac characteristics include cardiac geometry, ablation locations, action potential and conduction velocity, source location, arrhythmia type, and so on. In block 301, the component generates simulated cardiac anatomies, for example, as described in the '754 patent. In block 302, the component generates simulated electrical properties such as action potential and conduction velocity. A set of cardiac characteristics includes a simulated cardiac geometry, electrical properties, and so on. In block 303, the component selects an arrhythmia type (or no arrhythmia) such as roof AFL. In decision block 304, if all the arrhythmia types have already been selected, then the component completes, else the component continues at block 305. In block 305, the component selects the next set of cardiac characteristics. In decision block 306, if all the sets of cardiac characteristics have already been selected, then the component loops to block 303 to select the next arrhythmia type, else the component continues at block 307. In block 307, the component adds characteristics of the arrhythmia type to the cardiac characteristics. For example, for a roof AFL, the component modifies the electrical properties of the left atrium (represented by a 3D mesh) to model a reentrant circuit with a pathway between the left and right pulmonary veins. In block 308, the component runs a simulation of electrical activity of a heart assuming the set of cardiac characteristics. The simulation may represent, for example, five seconds of simulated electrical activity. In block 309, the component generates an ECG based on the simulated electrical activity. The ECG may be further based on thoracic characteristics such as body fat distribution. For each simulation, the component may generate an ECG assuming different thoracic characteristics. In block 310, the component adds to an AFL mapping library a mapping of the ECG (or ECGs based on different thoracic characteristics) to the set of cardiac characteristics and arrhythmia type.

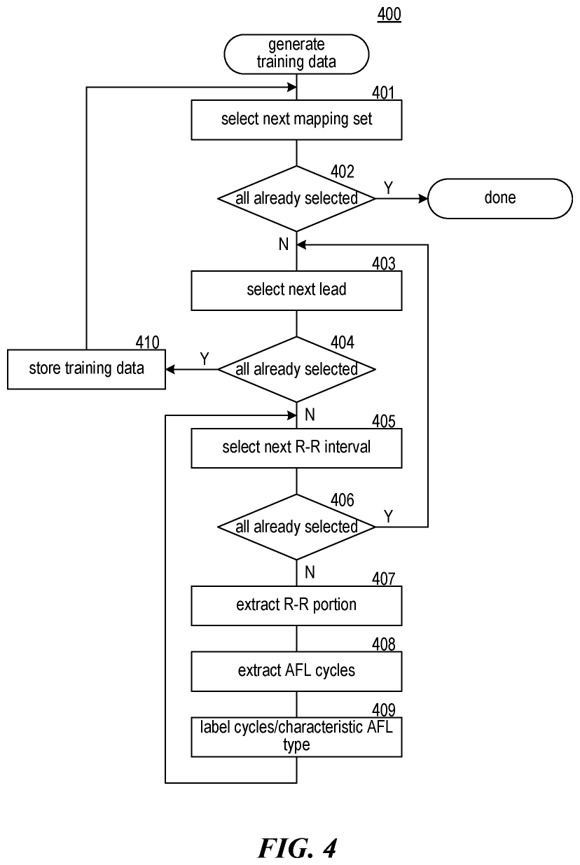

FIG. 4 is a flow diagram that illustrates a generate training data component that generates training data for the AFL ML model in some embodiments. The generate training data component 400 generates the training data from an AFL mapping library that includes mapping sets of ECGs, additional features, and AFL type that may be derived from simulations and/or clinical data. In block 401, the component selects the next mapping set. In decision block 402, if all the mapping sets have already been selected, then the component completes, else the component continues at block 403. In block 403, the component selects the next lead of the ECG of the selected mapping set. In decision block 404, if all the leads have already been selected, then the component continues at block 410, else the component continues at block 405. In block 405, the component selects the next R-R interval of the selected lead. In decision block 406, if all the R-R intervals of the selected lead have already been selected, then the component loops to block 403 to select the next lead, else the component continues at block 407. In block 407, the component extracts an R-R portion from the selected R-R interval. The selected R-R portion may represent a fixed portion before an R peak, the entire R-R interval, and so on. In block 408, the component extracts AFL cycles from the R-R portion. In block 409, the component labels the AFL cycles (and optionally in combination with additional features) with the AFL type of the selected mapping set and then loops to block 405 to select the next R-R interval. The AFL cycles may be normalized to a standard length such as 100 milliseconds. The source location of an AFL may be somewhat independent of the length of an AFL cycle. In block 408, the component stores the training data for the selected mapping set and loops to block 401 to select the next mapping set.

After the training data is generated, the AFL classification system trains the AFL ML model. Referring to FIG. 2, the AFL ML model may be trained using features that include an ECG (or a portion of an ECG) and additional features with a label relating to arrhythmia type. The arrhythmia ML sub-model may be trained using ECGs and additional features labeled as representing normal sinus rhythm or an arrhythmia. The atrial ML sub-model may be trained using arrhythmia ECGs and additional features labeled as representing an atrial or ventricular arrhythmia. The regular atrial ML sub-model may be trained using atrial ECGs and additional features labeled as representing regular or not regular atrial arrhythmias. The macro-reentrant AFL ML sub-model may be trained using regular ECGs and additional features with labels indicating macro-reentrant AFL or not. The left/right atrial ML sub-model may be trained using macro-reentrant AFL ECGs and additional features with labels indicating whether the source location of the AFL is in the left or right atrium. The MA/roof AFL ML sub-model may be trained using left atrial ECGs and additional features labeled as representing a mitral annular AFL or roof AFL. The typical CTI AFL ML sub-model may be trained using right atrial ECGs and additional features labeled as representing a typical CTI AFL or other types of right atrium AFL. Each ML sub-model may be trained using the same set of additional features. Alternatively, some ML sub-models may be trained using different additional features that are selected based on the classification performed by the ML sub-model. For example, an ablation location in the left ventricle may be an additional feature for the atrial ML sub-model but not for the right AFL ML sub-model.

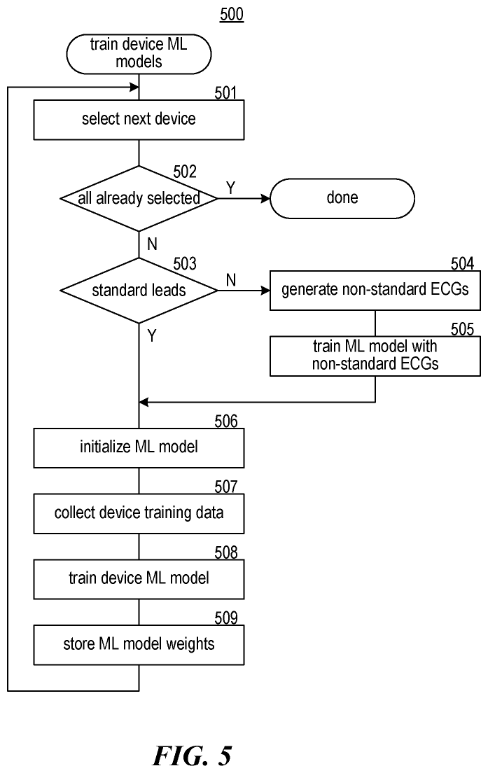

FIG. 5 is a flow diagram that illustrates a train device AFL ML models component in some embodiments. The train device AFL ML models component 500 is invoked to train device-specific AFL ML models for various devices such as different models of smartwatches, tablets, portable ECG devices, and so on. In block 501, the component selects the next device. In decision block 502, if all the devices have already been selected, then the component completes, else the component continues at block 503. In decision block 503, if the selected device generates ECGs with only standard leads, then the component continues at block 506, else the component continues at block 504. In block 504, the component generates non-standard ECGs, for example, from the simulations of electrical activity. The non-standard leads may be generated assuming non-standard electrode placements and non-standard combinations of readings from multiple electrodes used by the device. In block 505, the component trains an AFL ML model with non-standard ECGs and additional features labeled with arrhythmia type. In block 506, the component optionally initializes weights of the device-specific AFL ML model to weights of a previously trained AFL ML model such as the weights of an AFL ML model trained using standard leads. In block 507, the component collects training data of the device such as 3-lead ECGs and additional features. In block 508, the component trains the device-specific AFL ML model. In block 509, the component stores the weights of the device-specific AFL ML model and then loops to block 501 to select the next device.

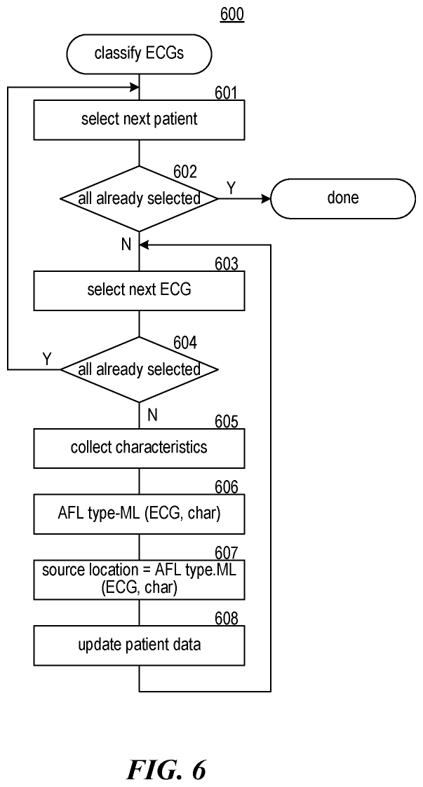

FIG. 6 is a flow diagram that illustrates a classify ECG component of the AFL classification system in some embodiments. The classify ECG component 600 is invoked to classify an ECG or other cardiogram of a patient. The component is provided an ECG and optionally additional features. In block 601, the component selects the next patient whose ECG is to be classified. In decision block 602, if all the patients have already been selected, then the component completes, else the component continues at block 603. In block 603, the component selects the next ECG of the patient. In decision block 604, if all the ECGs have already been selected, then the component loops to block 601 to select the next patient, else the component continues at block 605. In block 605, the component collects additional features of the patient. In block 606, the component invokes the AFL ML model passing an indication of the ECG and the additional features to identify the AFL type (or other arrhythmia type) represented by the ECG. In block 607, the component invokes a mapping ML model that inputs the ECG and outputs the source location of the AFL. A separate mapping ML model may be trained for each AFL type. In block 608, the component updates the patient data to indicate the AFL type (or other arrhythmia type) and source location and then loops to block 603 to select the next ECG of the selected patient.

The following paragraphs describe various aspects of the AFL classification system. An implementation of the AFL classification system may employ any combination or sub-combination of the aspects and may employ additional aspects. The processing of the aspects may be performed by one or more computing systems with one or more processors that execute computer-executable instructions that implement the aspects and that are stored on one or more computer-readable storage mediums.

In some aspects, the techniques described herein relate to one or more computing systems for generating a machine learning (ML) model having ML sub-models to identify an atrial flutter (AFL) type of a cardiac arrhythmia, the one or more computing systems including: one or more computer-readable storage mediums that store: mappings that each maps a cardiogram to an arrhythmia sub-classification that the cardiogram represents, the arrhythmia sub-classifications based on levels of a hierarchy of arrhythmia sub-classification types, the arrhythmia sub-classification types of the lowest level being AFL types; and computer-executable instructions for controlling the one or more computing systems to: for each sub-classification type, generate training data for that sub-classification type that includes, for each of a plurality of the mappings, a feature vector with one or more features derived from that mapping and a label indicating whether the feature vector represents that sub-classification type or does not represent that sub-classification type; and train an ML sub-model for that sub-classification type using the training data for that sub-classification type to learn weights for that ML sub-model, the ML sub-model for that sub-classification type for inputting a feature vector with one or more features derived from a cardiogram and outputting an AFL type; and store the weights of the ML model of that classification; and generate a patient feature vector derived from a patient cardiogram; and apply each ML sub-model in hierarchical order to determine whether the patient cardiogram represents an AFL and if so, identify the AFL type; and one or more processors for controlling the one or more computing systems to execute one or more of the computer-executable instructions. In some aspects, the techniques described herein relate to one or more computing systems wherein a cardiogram is represented as a voltage-time series. In some aspects, the techniques described herein relate to one or more computing systems wherein a cardiogram is represented as an image of an electrocardiogram. In some aspects, the techniques described herein relate to one or more computing systems wherein the one or more features of a feature vector are specific to the sub-classification level. In some aspects, the techniques described herein relate to one or more computing systems wherein a feature of a feature vector for an ML sub-model for an AFL type is an AFL cycle. In some aspects, the techniques described herein relate to one or more computing systems wherein features of a feature vector for an ML sub-model for an AFL type are based on one or more of an AFL cycle, an interval prior to an R peak, and an R-R interval.

In some aspects, the techniques described herein relate to one or more computing systems wherein the ML model includes ML sub-models to classify a cardiogram being arrhythmia cardiogram, an arrhythmia cardiogram as an atrial cardiogram, an atrial cardiogram as a regular atrial cardiogram, a regular atrial cardiogram as a macro-reentrant AFL cardiogram, a macro-reentrant AFL cardiogram as left atrial or right atrial cardiogram, a right atrial cardiogram as a typical AFL cardiogram, and a left atrial cardiogram as mitral annular AFL cardiogram or roof AFL cardiogram. In some aspects, the techniques described herein relate to one or more computing systems wherein classifications of one or more ML sub-models are combined into a combined ML sub-model. In some aspects, the techniques described herein relate to one or more computing systems wherein the mappings are based on simulations of electrical activity of a heart or based on clinical data. In some aspects, the techniques described herein relate to one or more computing systems wherein a feature is a representation of a heart. In some aspects, the techniques described herein relate to one or more computing systems wherein a feature relates to cardiac geometry. In some aspects, the techniques described herein relate to one or more computing systems wherein the AFL type is one or more of typical cavotricuspid isthmus (CTI) AFL, mitral annular AFL, and roof AFL. In some aspects, the techniques described herein relate to one or more computing systems wherein a cardiogram includes one or more leads. In some aspects, the techniques described herein relate to one or more computing systems wherein the ML model is based on one or more of a convolution neural network, a recurrent neural network, and decision tree. In some aspects, the techniques described herein relate to one or more computing systems wherein the mappings are generated based on simulation of electrical activity of an AFL.

In some aspects, the techniques described herein relate to a method performed by one or more computing systems for generating a device-specific machine learning (ML) model for identifying atrial flutter (AFL) type that is based on device cardiograms collected by a cardiogram collection device, the method including: accessing device mappings of device cardiograms collected by the cardiogram collection device to arrhythmia classification; accessing prior weights of prior ML model that is trained based mappings of prior cardiograms to an indication of AFL type, the prior cardiograms not collected by the cardiogram collection device; initializing device weights of the device ML model to the prior weights; training the device ML model to generate device weights using the device mappings of device cardiograms to AFL type as training data and the initialized device weights; and storing the generated device weights. In some aspects, the techniques described herein relate to a method further including wherein a prior cardiogram is generated based on a simulation of electrical activity of a heart assuming placement of electrodes used in collecting one or more leads of a device cardiogram. In some aspects, the techniques described herein relate to a method wherein the cardiogram collection device is employed in a non-clinical setting. In some aspects, the techniques described herein relate to a method wherein the cardiogram collection device is a smartwatch or a smartphone.

In some aspects, the techniques described herein relate to one or more computing systems for identifying a patient atrial flutter (AFL) type of an AFL of a patient, the one or more computing systems including: one or more computer-readable storage mediums that store computer-executable instructions for controlling the one or more computing systems to: receive a patient cardiogram collected from a patient; identify one or more AFL cycles of the patient cardiogram; for each identified AFL cycle, apply a machine learning (ML) model to that identified AFL cycle to identify an AFL type, the ML model having a hierarchy of ML sub-models of sub-classifications of an AFL; and identify a patient AFL type that is derived from identified AFL types; and output an indication of the patient AFL type; and one or more processors for controlling the one or more computing systems to execute one or more of the computer-executable instructions. In some aspects, the techniques described herein relate to one or more computing systems wherein the computer-executable instructions further include instructions to identify a source location of the AFL based on the patient cardiogram and the patient AFL type. In some aspects, the techniques described herein relate to one or more computing systems wherein the source location is identified based on mappings of cardiograms to source locations of AFLs of the patient AFL type. In some aspects, the techniques described herein relate to one or more computing systems wherein the source location is identified using a source location ML model that is trained based on the mappings. In some aspects, the techniques described herein relate to one or more computing systems wherein the AFL ML model is trained using simulated and/or clinical data. In some aspects, the techniques described herein relate to a method performed by one or more computing systems for identifying a patient atrial flutter (AFL) type of a patient, the method including: accessing a patient cardiogram collected from a patient; applying an AFL machine learning (ML) model that includes a hierarchy of ML sub-models to identify the patient AFL type, the AFL ML model trained using training cardiograms labeled with AFL type, the ML sub-model being applied in hierarchical order; and outputting an indication of the patient AFL type. In some aspects, the techniques described herein relate to a method further including identifying a source location of the AFL based on the patient cardiogram and the patient AFL type. In some aspects, the techniques described herein relate to a method wherein the source location is identified based on mappings of cardiograms to source locations of AFLs of the patient AFL type. In some aspects, the techniques described herein relate to a method wherein the source location is identified using a source location ML model that is trained based on the mappings.

In some aspects, the techniques described herein relate to a method performed by one or more computing systems for simulating electrical activity of an atrial flutter, the method including: designating an area of slow conduction electrical activity in an atrium in which electrical activity is conducted slower than in a surrounding area in the atrium; designating a line of block adjacent to the area of slow conduction to block conduction of electrical activity through the line of block; initiating running of a first simulation to simulate electrical activity based on an electrical stimulus being applied on the side of line of block that is opposite the area of slow conduction; during the running of the first simulation, removing the line of block to allow conduction of electrical activity through the area of the removed line of block; continuing running the first simulation wherein electrical activity is conducted through the area of the removed line of block; terminating the running of the simulation after the atrial flutter has stabilized; initializing running of a second simulation by initializing electrical activity of the second simulation based on the electrical activity of the first simulation after the atrial flutter has stabilized; and running the second simulation until the atrial flutter has stabilized. In some aspects, the techniques described herein relate to a method wherein the running of the simulation is based on a three-dimensional (3D) mesh representing geometry of the atrium, the 3D mesh specified by vertices and edges connecting the vertices, each vertex associated with electrical characteristics and values representing electrical activity at the vertex. In some aspects, the techniques described herein relate to a method wherein the running of the simulation includes updating values of vertices for a plurality of simulation intervals. In some aspects, the techniques described herein relate to a method further including generating a cardiogram based on the simulated electrical activity of the simulation. In some aspects, the techniques described herein relate to a method wherein the cardiogram is a vectorcardiogram. In some aspects, the techniques described herein relate to a method wherein the area of slow conduction represents the source location of the atrial flutter. In some aspects, the techniques described herein relate to a method further including running a plurality of simulations assuming different atrial geometries, atrial electrical characteristics, areas of slow conduction, and/or lines of block. In some aspects, the techniques described herein relate to a method further including: for each simulation, generating a cardiogram based on the simulated electrical activity of the simulation; and generating a training data set of training data that includes the generated cardiogram labeled with a location of the area of slow conduction; and training a machine learning (ML) model using the training data, wherein the trained ML model inputs a cardiogram and outputs a location corresponding to a source location of the atrial flutter. In some aspects, the techniques described herein relate to a method further including receiving a patient cardiogram of a patient and inputting the patient cardiogram into the trained ML model to output a source location of the atrial flutter represented by the patient cardiogram. In some aspects, the techniques described herein relate to a method wherein the ML model is based on a neural network. In some aspects, the techniques described herein relate to a method further including designating an atrial flutter type for the atrial flutter based on the location of the designated area of slow conduction and the location of the designated line of block. In some aspects, the techniques described herein relate to a method further including: for each simulation, generating a cardiogram based on the simulated electrical activity of the simulation; and generating a training data set of training data that includes a generated cardiogram labeled with the designated atrial flutter type of the simulation; and training a machine learning (ML) model using the training data, wherein trained ML model inputs a cardiogram and outputs an atrial flutter type. In some aspects, the techniques described herein relate to a method further including receiving a patient cardiogram of a patient and inputting the patient cardiogram into the trained ML model to output the atrial flutter type represented by the patient cardiogram. In some aspects, the techniques described herein relate to a method wherein the ML model is based on a neural network. In some aspects, the techniques described herein relate to a method wherein the atrial flutter is typical cavotricuspid isthmus atrial flutter and the area of slow conduction is in the cavotricuspid isthmus. In some aspects, the techniques described herein relate to a method wherein the atrial flutter is a mitral annular atrial flutter and the area of slow conduction is around the mitral annulus. In some aspects, the techniques described herein relate to a method wherein the atrial flutter is a roof atrial flutter and the area of slow conduction is in the pathway between the left pulmonary vein and the right pulmonary vein.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims.

1. One or more computing systems for generating a machine learning (ML) model having ML sub-models to identify an atrial flutter (AFL) type of a cardiac arrhythmia, the one or more computing systems comprising:
one or more computer-readable storage mediums that store:
mappings that each maps a cardiogram to an arrhythmia sub-classification that the cardiogram represents, the arrhythmia sub-classifications based on levels of a hierarchy of arrhythmia sub-classification types, the arrhythmia sub-classification types of the lowest level being AFL types; and
computer-executable instructions for controlling the one or more computing systems to:
for each sub-classification type,
generate training data for that sub-classification type that includes, for each of a plurality of the mappings, a feature vector with one or more features derived from that mapping and a label indicating whether the feature vector represents that sub-classification type or does not represent that sub-classification type; and
train an ML sub-model for that sub-classification type using the training data for that sub-classification type to learn weights for that ML sub-model, the ML sub-model for that sub-classification type for inputting a feature vector with one or more features derived from a cardiogram and outputting an AFL type; and
store the weights of the ML model of that classification; and
generate a patient feature vector derived from a patient cardiogram; and
apply each ML sub-model in hierarchical order to determine whether the patient cardiogram represents an AFL and if so,
identify the AFL type; and
one or more processors for controlling the one or more computing systems to execute one or more of the computer-executable instructions.

2. The one or more computing systems of claim 1 wherein a cardiogram is represented as a voltage-time series.

3. The one or more computing systems of claim 1 wherein a cardiogram is represented as an image of an electrocardiogram.

4. The one or more computing systems of claim 1 wherein the one or more features of a feature vector are specific to the sub-classification level.

5. The one or more computing systems of claim 4 wherein a feature of a feature vector for an ML sub-model for an AFL type is an AFL cycle.

6. The one or more computing systems of claim 4 wherein features of a feature vector for an ML sub-model for an AFL type are based on one or more of an AFL cycle, an interval prior to an R peak, and an R-R interval.

7. The one or more computing systems of claim 1 wherein the ML model includes ML sub-models to classify a cardiogram being arrhythmia cardiogram, an arrhythmia cardiogram as an atrial cardiogram, an atrial cardiogram as a regular atrial cardiogram, a regular atrial cardiogram as a macro-reentrant AFL cardiogram, a macro-reentrant AFL cardiogram as left atrial or right atrial cardiogram, a right atrial cardiogram as a typical AFL cardiogram, and a left atrial cardiogram as mitral annular AFL cardiogram or roof AFL cardiogram.

8. The one or more computing systems of claim 7 wherein classifications of one or more ML sub-models are combined into a combined ML sub-model.

9. The one or more computing systems of claim 1 wherein the mappings are based on simulations of electrical activity of a heart or based on clinical data.

10. The one or more computing systems of claim 1 wherein a feature is a representation of a heart.

11. The one or more computing systems of claim 1 wherein a feature relates to cardiac geometry.

12. The one or more computing systems of claim 1 wherein the AFL type is one or more of typical cavotricuspid isthmus (CTI) AFL, mitral annular AFL, and roof AFL.

13. The one or more computing systems of claim 1 wherein a cardiogram comprises one or more leads.

14. The one or more computing systems of claim 1 wherein the ML model is based on one or more of a convolution neural network, a recurrent neural network, and decision tree.

15. The one or more computing systems of claim 1 wherein the mappings are generated based on simulation of electrical activity of an AFL.

16. A method performed by one or more computing systems for generating a device-specific machine learning (ML) model for identifying atrial flutter (AFL) type that is based on device cardiograms collected by a cardiogram collection device, the method comprising:
accessing device mappings of device cardiograms collected by the cardiogram collection device to arrhythmia classification;
accessing prior weights of prior ML model that is trained based mappings of prior cardiograms to an indication of AFL type, the prior cardiograms not collected by the cardiogram collection device;
initializing device weights of the device ML model to the prior weights;
training the device ML model to generate device weights using the device mappings of device cardiograms to AFL type as training data and the initialized device weights; and
storing the generated device weights.

17. The method of claim 16 further comprising wherein a prior cardiogram is generated based on a simulation of electrical activity of a heart assuming placement of electrodes used in collecting one or more leads of a device cardiogram.

18. The method of claim 16 wherein the cardiogram collection device is employed in a non-clinical setting.

19. The method of claim 16 wherein the cardiogram collection device is a smartwatch or a smartphone.

20. One or more computing systems for identifying a patient atrial flutter (AFL) type of an AFL of a patient, the one or more computing systems comprising:
one or more computer-readable storage mediums that store computer-executable instructions for controlling the one or more computing systems to:
receive a patient cardiogram collected from a patient;
identify one or more AFL cycles of the patient cardiogram;
for each identified AFL cycle, apply a machine learning (ML) model to that identified AFL cycle to identify an AFL type, the ML model having a hierarchy of ML sub-models of sub-classifications of an AFL; and
identify a patient AFL type that is derived from identified AFL types; and
output an indication of the patient AFL type; and
one or more processors for controlling the one or more computing systems to execute one or more of the computer-executable instructions.

21. The one or more computing systems of claim 20 wherein the computer-executable instructions further include instructions to identify a source location of the AFL based on the patient cardiogram and the patient AFL type.

22. The one or more computing systems of claim 21 wherein the source location is identified based on mappings of cardiograms to source locations of AFLs of the patient AFL type.

23. The one or more computing systems of claim 22 wherein the source location is identified using a source location ML model that is trained based on the mappings.

24. The one or more computing systems of claim 20 wherein the AFL ML model is trained using simulated and/or clinical data.

25. A method performed by one or more computing systems for identifying a patient atrial flutter (AFL) type of a patient, the method comprising:
accessing a patient cardiogram collected from a patient;
applying an AFL machine learning (ML) model that includes a hierarchy of ML sub-models to identify the patient AFL type, the AFL ML model trained using training cardiograms labeled with AFL type, the ML sub-model being applied in hierarchical order; and
outputting an indication of the patient AFL type.

26. The method of claim 25 further comprising identifying a source location of the AFL based on the patient cardiogram and the patient AFL type.

27. The method of claim 26 wherein the source location is identified based on mappings of cardiograms to source locations of AFLs of the patient AFL type.

28. The method of claim 27 wherein the source location is identified using a source location ML model that is trained based on the mappings.

29. A method performed by one or more computing systems for simulating electrical activity of an atrial flutter, the method comprising:
designating an area of slow conduction electrical activity in an atrium in which electrical activity is conducted slower than in a surrounding area in the atrium;
designating a line of block adjacent to the area of slow conduction to block conduction of electrical activity through the line of block;
initiating running of a first simulation to simulate electrical activity based on an electrical stimulus being applied on the side of line of block that is opposite the area of slow conduction;
during the running of the first simulation, removing the line of block to allow conduction of electrical activity through the area of the removed line of block;
continuing running the first simulation wherein electrical activity is conducted through the area of the removed line of block;
terminating the running of the simulation after the atrial flutter has stabilized;
initializing running of a second simulation by initializing electrical activity of the second simulation based on the electrical activity of the first simulation after the atrial flutter has stabilized; and
running the second simulation until the atrial flutter has stabilized.

30. The method of claim 29 wherein the running of the simulation is based on a three-dimensional (3D) mesh representing geometry of the atrium, the 3D mesh specified by vertices and edges connecting the vertices, each vertex associated with electrical characteristics and values representing electrical activity at the vertex.

31. The method of claim 30 wherein the running of the simulation includes updating values of vertices for a plurality of simulation intervals.

32. The method of claim 29 further comprising generating a cardiogram based on the simulated electrical activity of the simulation.

33. The method of claim 32 wherein the cardiogram is a vectorcardiogram.

34. The method of claim 29 wherein the area of slow conduction represents the source location of the atrial flutter.

35. The method of claim 29 further comprising running a plurality of simulations assuming different atrial geometries, atrial electrical characteristics, areas of slow conduction, and/or lines of block.

36. The method of claim 35 further comprising:
for each simulation,
generating a cardiogram based on the simulated electrical activity of the simulation; and
generating a training data set of training data that includes the generated cardiogram labeled with a location of the area of slow conduction; and
training a machine learning (ML) model using the training data, wherein the trained ML model inputs a cardiogram and outputs a location corresponding to a source location of the atrial flutter.

37. The method of claim 36 further comprising receiving a patient cardiogram of a patient and inputting the patient cardiogram into the trained ML model to output a source location of the atrial flutter represented by the patient cardiogram.

38. The method of claim 36 wherein the ML model is based on a neural network.

39. The method of claim 29 further comprising designating an atrial flutter type for the atrial flutter based on the location of the designated area of slow conduction and the location of the designated line of block.

40. The method of claim 39 further comprising:
for each simulation,
generating a cardiogram based on the simulated electrical activity of the simulation; and
generating a training data set of training data that includes a generated cardiogram labeled with the designated atrial flutter type of the simulation; and
training a machine learning (ML) model using the training data, wherein trained ML model inputs a cardiogram and outputs an atrial flutter type.

41. The method of claim 40 further comprising receiving a patient cardiogram of a patient and inputting the patient cardiogram into the trained ML model to output the atrial flutter type represented by the patient cardiogram.

42. The method of claim 41 wherein the ML model is based on a neural network.

43. The method of claim 29 wherein the atrial flutter is typical cavotricuspid isthmus atrial flutter and the area of slow conduction is in the cavotricuspid isthmus.

44. The method of claim 29 wherein the atrial flutter is a mitral annular atrial flutter and the area of slow conduction is around the mitral annulus.

45. The method of claim 29 wherein the atrial flutter is a roof atrial flutter and the area of slow conduction is in the pathway between the left pulmonary vein and the right pulmonary vein.
